(19) United States
(12) Patent Application Publication
(10) Pub. No.: US 2022/0339475 A1
(43) Pub. Date: Oct. 27, 2022

(54) Mask and Removable Cidal Metal or Cidal Metal Alloy Insert

(71) Applicant: Phyllis Kuhn, Redington Shores, FL (US)

(72) Inventor: Phyllis Kuhn, Redington Shores, FL (US)

(21) Appl. No.: 17/862,188

(22) Filed: Jul. 11, 2022

Related U.S. Application Data
(63) Continuation-in-part of application No. PCT/US2021/013783, filed on Jan. 17, 2021.
(60) Provisional application No. 62/962,495, filed on Jan. 17, 2020.

(51) Int. Cl. A62B 23/02; B32B 5/02; B32B 15/14; B32B 7/12; B32B 7/06; B01D 39/10; B01D 46/52; B01D 46/00; B32B 15/02

(52) U.S. Cl. CPC A62B 23/025; B32B 5/028; B32B 15/14; B32B 7/12; B32B 7/06; B01D 39/10; B01D 46/521; B01D 46/0028; B32B 15/02; B32B 2437/00; B01D 2265/04; B01D 2265/02; B01D 2239/1233; B01D 2239/0442; B32B 2262/103; B32B 2262/16; B32B 2307/724; B32B 2571/00

(57) ABSTRACT

A face mask and a removable insert, the insert being constructed primarily of cidal metal or cidal metal alloy wherein the cidal metal or cidal metal alloy is the major structural component of the insert. The insert is positioned on the face mask to cover at least a portion of the wearer's mouth and nose when the mask is worn on the wearer's face. The insert has a filtering portion comprising cidal metal mesh or cidal metal alloy mesh to provide cidal action, air purification, and self-disinfection.

### CROSS REFERENCE TO RELATED APPLICATIONS

This is a U.S. Bypass Continuation-in-Part Application of Patent Cooperation Treaty (PCT) Application PCT/US2021/013783, filed Jan. 17, 2021, which takes priority from and claims the benefit of U.S. Provisional Patent Application 62/962,495, filed Jan. 17, 2020, both prior Applications being incorporated herein by reference.

### BACKGROUND OF THE INVENTION

With the rise of the worldwide Covid-19 pandemic, the use of facemasks has grown exponentially in most countries. However, even before this global health crisis, facemasks with filtration capabilities had been frequently worn for a broad range of purposes and applications. Such masks can include disposable facemasks, such as those cleared by the U.S. Food and Drug Administration (FDA) for use as medical devices and devices worn by medical professionals, single and multiple use masks such as dust masks and respirators used in industry and by home consumers, rigid and multi-use masks, and numerous other types used for different environments and circumstances. Some masks are labeled for specific applications such as surgical, dental, medical procedure, isolation, and laser masks.

Such facemasks have several designs. One type is cloth, woven, or flexible material affixed to a wearer's head with one or two ties or elastic bands, conforming to the face with the aid of a flexible adjustment for the nose bridge, and may be flat/pleated or duck-billed in shape. Another type of facemask is pre-molded or pre-formed, adheres to the head with a single or double elastic band, and has a flexible adjustment for the nose bridge. A third type is flat/pleated and affixes to the head with ear loops. Respirator-type masks often include removable or replaceable filters and/or exhale valves.

Facemasks cleared by the FDA for use as medical devices have been determined to have specific levels of protection from penetration of blood and body fluids. Facemasks often help stop droplets from being spread by the person wearing them. They are often also used to keep splashes or sprays from reaching the mouth and nose of the facemask wearer, but are often not intended to protect against very small particle aerosols.

Cidal (pathogen and microbial-killing) metals, such as but not limited to copper, silver, gold, and related alloys such as bronze and brass, are often incorporated into the cotton, woven organic, or polymer fabric structural material of a conventional woven or fiber facemask to improve cidal action and air purification due to cidal (killing) antimicrobial properties of such metals. In some cases, cidal solutions can also be applied to the conventional structural mask material. However, even with the application of such cidal substances, the main structural materials of conventional facemasks still present significant problems for wearers.

In conventional woven or fiber masks, the cotton, woven organic, or polymer fabric major structural material does not normally provide a physical barrier to water or blood. Rather, such materials generally exhibit wicking which actually promotes the penetration of water or blood, regardless of whether the water or blood is splashed or poured on to the mask. While bacteria, viruses, and other pathogens often require water droplets to travel through the air, wicking permits bacteria and viruses to penetrate the mask, reducing the mask's filtering effectiveness.

Woven or fiber masks are also often single use and attempts to disinfect such masks, such as through the process of autoclaving, may have adverse effects on a mask's major structural material by weakening or altering its individual fiber or woven properties. Thus, disposal after a single use often becomes necessary. If cidal metal materials have been interwoven into the fibers of such masks, those materials are lost during disposal which is wasteful and less environmentally friendly.

### SUMMARY OF THE INVENTION

A removable insert for a face mask includes an insert body constructed primarily of a material that includes a cidal metal or cidal metal alloy. The cidal metal or cidal metal alloy is also the major structural component of the insert body. The insert is positioned on the face mask to allow the insert body to cover at least a portion of the wearer's mouth, nose, or mouth and nose when the mask is worn on the wearer's face. The insert can be removed from the mask for disinfection and/or later re-use with the same or a different mask.

The insert includes a filtering portion also comprising cidal metal mesh or cidal metal alloy mesh. The cidal metal or cidal metal alloy mesh provides cidal action, air purification, and self-disinfection of the insert. In some contemplated embodiments, the cidal metal or cidal metal alloy mesh of the filtering portion has an average wire diameter and an average width of opening that is sufficiently small to prevent, due to water or blood viscosity, the penetration of water or blood through the filtering portion. However, to allow for additional mask sanitizing and reuse, the filtering portion mesh also has an average wire diameter and an average opening width of sufficient size to allow penetration of disinfecting solution due to disinfecting solution viscosity that is less than water or blood viscosity.

In some embodiments, the use of cidal metal or cidal metal alloy in the insert body allows for alternative means of additional mask sanitizing through methods such as heating or autoclaving.

### BRIEF DESCRIPTION OF THE DRAWINGS

For a more complete understanding and appreciation of this invention, and its many advantages, reference will be made to the following Detailed Description of the Invention taken in conjunction with the accompanying drawings.

FIG. 1 is front view of a removable insert according to one contemplated embodiment of the invention;

FIG. 2A is rear perspective view of the insert of FIG. 1 and a face mask according to the invention;

FIG. 2B is a rear view of the insert of FIGS. 1 and 2A attached to the face mask of FIG. 2A;

FIG. 3 is front view of a removable insert according to one contemplated embodiment of the invention;

FIG. 4 is front view of a removable insert according to one contemplated embodiment of the invention;

FIG. 5A is rear perspective view of an insert and a face mask according to one contemplated embodiment of the invention;

FIG. 5B is a rear perspective view of the insert of FIG. 5A attached to the face mask of FIG. 5A;

FIG. 6A is front exploded perspective view of an insert and a face mask according to one contemplated embodiment of the invention;

FIG. 6B is a front perspective view of the insert of FIG. 6A attached to the face mask of FIG. 6A;

FIG. 7 is front view of a removable insert according to one contemplated embodiment of the invention;

FIG. 8 is front view of a removable insert according to one contemplated embodiment of the invention;

FIG. 9A is a front view of a face mask according to one contemplated embodiment of the invention;

FIG. 9B is a rear view of a removable insert attached to the face mask of FIG. 9A according to one contemplated embodiment of the invention;

FIG. 10A is a front view of a face mask according to one contemplated embodiment of the invention;

FIG. 10B is a rear view of a removable insert attached to the face mask of FIG. 10A according to one contemplated embodiment of the invention;

FIG. 11 is a rear view of a removable insert according to one contemplated embodiment of the invention;

FIG. 12 is a rear view of the removable insert of FIG. 11 after modification;

FIG. 13A is a rear view of a top attachment strip according to one embodiment of the invention;

FIG. 13B is a rear view of the top attachment strip of FIG. 13A; and

FIG. 14 is a front view of the rear attachment strip of FIG.13B attached to the front of the removable insert of FIG. 12.

### DETAILED DESCRIPTION OF THE INVENTION

Referring to the drawings, some reference numerals are used to designate the same or corresponding parts through several of the embodiments and figures shown and described. Variations in corresponding parts are denoted in specific embodiments with the addition of lowercase letters. Subsequent variations in components that are depicted in the figures but not described are intended to correspond to the specific embodiments mentioned earlier and are discussed to the extent that they vary in form or function. It will be understood generally that variations in the embodiments could be interchanged without deviating from the intended scope of the invention.

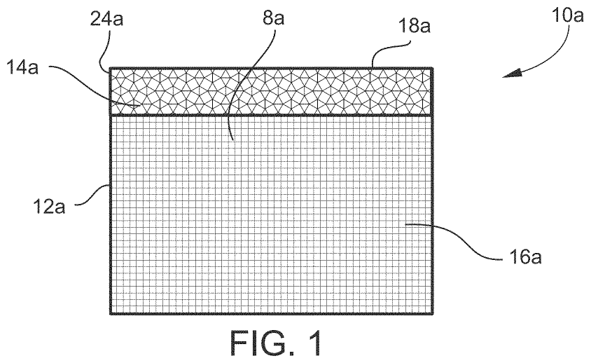

FIG. 1 depicts a front view with visible front side 8a of a removable insert 10a of the invention having an insert body 12a and a top attachment strip 14a. The insert body 12a includes a filtering portion 16a that is a flat and flexible piece of copper mesh. As best understood from FIG. 1, the filtering portion 16a comprises the entirety of the insert body 12a such that the copper from which it is fabricated is the major structural component of the inset body 12a. The top attachment strip 14a is positioned on the front side 8a near the top edge 18a of the insert body 12a to minimize interference with airflow while allowing the insert 10a to attach to and remain removably attached to a face mask. The attachment strip 14a can include a conventional adhesive such as glue or rubber cement, a mechanical or hook attachment such as a VELCRO™ strip, or any other non-permanent arrangement allowing for adhesion or mechanical positioning of the insert 10a.

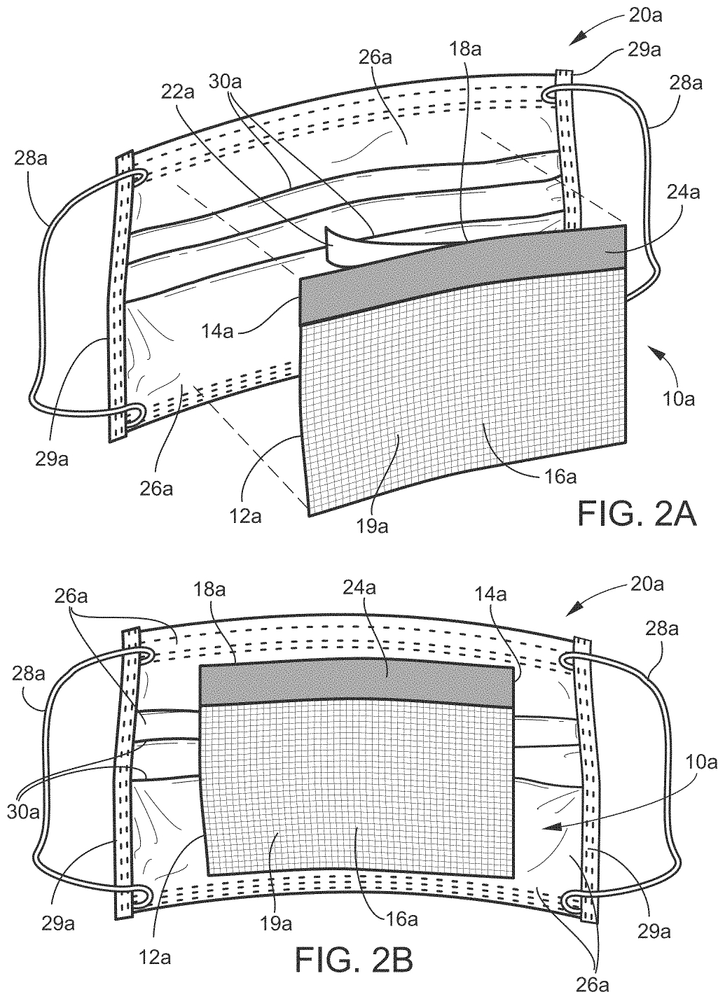

Referring now to FIG. 2A, which depicts rear views of the insert 10a of FIG. 1 with visible rear side 19a and a conventional flat face mask 20a just prior to insert attachment, and FIG. 2B, which depicts a rear view of the insert 10a attached to the face flat face mask 20a, a protective covering 22a of the attachment strip 14a may be removed as shown in FIG. 2A prior to mask attachment. A strip of soft moleskin 24a is positioned near the top edge 18a on the rear side 19a of the insert body 12a for additional padding against the wearer's face while minimizing interference with air flow through the filtering portion 16a.

With further reference to FIGS. 2A and 2B, the conventional flat face mask 20a includes a cotton or woven fiber mask body 26a with elastic ear straps 28a anchored at side folds 29a to allow a wearer to position the mask 20a completely over the wearer's mouth and nose. Mask pleats 30a extend the width of the mask 20a to allow the otherwise flat shape of the mask 20a to extendably fit over the over the wearer's facial features from the chin to well above the nostrils of the wearer's nose.

As a cidal metal, the copper of the filtering portion 16a and insert body 12a is capable of killing most pathogens and microorganisms but is not harmful to humans. As the wearer breathes through the insert, the mesh of the filtering portion 16a releases copper ions that can adhere to the wearer's mucus linings and help prevent some illnesses such as colds. The copper mesh 28a of the insert body 12a is also highly effective for filtering out most small particulate matter. Further, copper possesses anti-inflammatory properties and is potentially capable of shrinking inflamed nasal membranes and sinus swelling, possibly increasing user comfort. In this conceptual example of FIGS. 1 through 2B, the insert body 12a is copper mesh having an approximate wire diameter of 0.0045 inches and width opening of 0.00555 inches with approximately 30.3% open area and with approximately 100×100 mesh per linear inch, such as item #100×100 0.0045 cu or similar item available from the Belleville Wire Cloth Company of Cedar Grove, N.J.

With further reference to FIGS. 1 through 2B, the copper mesh of the filtering portion 16a forms the insert body 12a. Thus, the copper material of the mesh is itself the major structural component of the insert 10a. Although this illustrative example utilizes copper as the major structural component of the insert 10a, it will be appreciated that other cidal metals or cidal metal alloys such silver, gold, bronze, brass, and more exotic cidal alloys can also be used as the major structural component of the insert 10a within the contemplated scope of the current invention. Such cidal metals or cidal metal alloys can also be used as filtering portions of an insert within the contemplated scope of the invention where the filtering portion does not extend the entire width or to the entire dimensional extent of the insert.

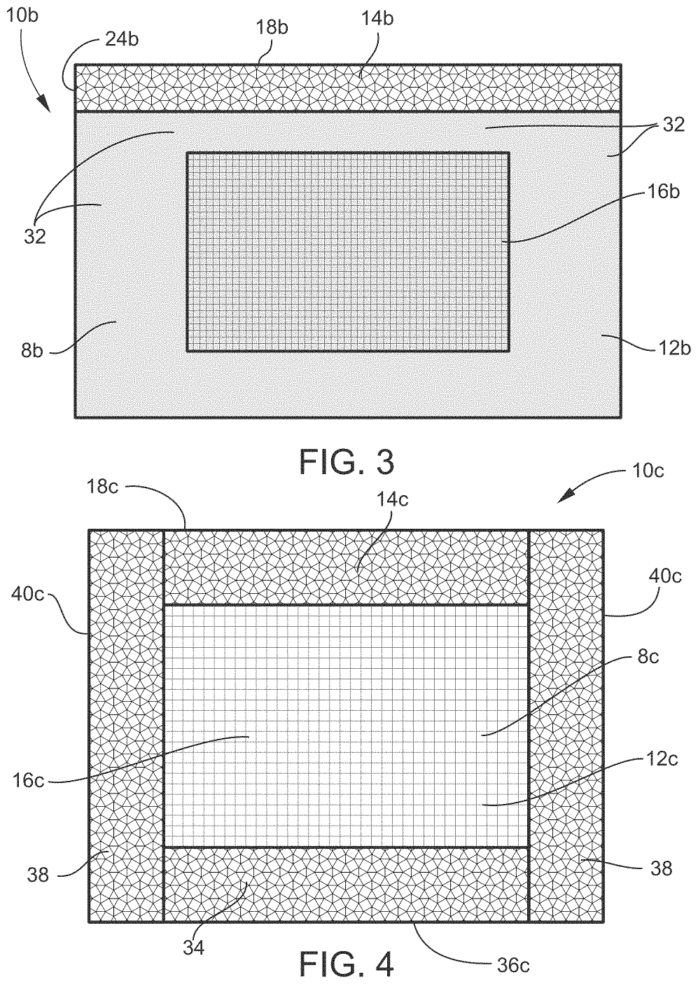

For example, with quick reference to FIG. 3, another contemplated insert 10b of the invention includes an insert body 12b having a filtering portion 16b of cidal metal or cidal metal alloy mesh that does not extend across the entire surface of the insert body 12b. A non-mesh area 32 of non-mesh cidal metal or cidal metal alloy surrounds all four sides of the mesh filtering portion 16b to occupy a significant portion of the flat surface of the insert body 12b. However, the filtering portion 16b still occupies a sufficient portion of the flat surface of the insert body 12b to allow for sufficient air flow to permeate and pass through the insert 10b during use.

Referring again to FIGS. 1 through 2A, the copper mesh of the insert body 12a is hydrophobic such that poured water and water droplets tend to not penetrate the mask due to natural water tension and typical water viscosity. Blood is approximately eight times as viscid as water. Thus, water or blood applied to the insert body 12a tends to bead up rather than passing through or being absorbed into the copper mesh of the filtering portion 16a. The illustrated example of FIGS. 1 through 2B contemplates copper mesh in the insert 10a having an approximate wire diameter of 0.0045 inches and approximate width opening of 0.00555 inches and with approximately 100×100 mesh per linear inch. It will be appreciated that some preferred embodiments utilize mesh with similar water-repelling hydrophobic characteristics. Meshes with wire diameters of approximately 0.0014 to 0.0045 inches and approximate width openings of 0.00170 to 0.00555 inches and with approximately 100×100 to 325×325 mesh per linear inch are likely to exhibit similar hydrophobic characteristics. It is further contemplated any such cidal metal mesh or cidal metal alloy mesh with a wire diameter less than approximately 0.0100 inches, and preferably less than approximately 0.0070 inches, could be appropriately implemented.

Although such ranges repel and resist wicking penetration of water or blood alone, such ranges also allow for sanitizing, subsequent reuse of inserts with disinfecting agents, and easy breathing by the wearer such that air flows through the mesh and not in or out of gaps. For example, low isopropyl alcohol viscosity would allow for penetration of the copper mesh of the filtering portion 16a of the insert 10a of FIGS. 1 through 2B and therefore allow the use of alcohol for insert disinfection. A 70% isopropyl alcohol solution would have a lower viscosity than water or blood, also permitting the use of such a solution as a disinfectant for the same insert 10a. These structural disinfection advantages would be in addition to the natural disinfection that would occur and be ongoing due to both the filtering portion 16a and major structural component/insert body 12a being cidal copper. In some embodiments, it could be further advantageous to effect additional disinfecting or sterilizing by heating or autoclaving an insert. For example, in FIGS. 1 through 2B, the insert 10a might be heated or autoclaved after removal from the mask 20a.

Although the invention has been shown and described as having single top attachment strip for positioning and attaching an insert of the invention to a face mask, it will be appreciated that other means of attachment are also possible within the intended scope of the invention. For example, FIG. 4 depicts a front view of an insert 10c having an insert body 12c that, along with a top attachment strip 14c near the top edge 18c, also includes a bottom attachment strip 34 near the bottom edge 36c of the insert body 12c and side attachment strips 38 near the side edges 40c of the insert body 12c. Like the top attachment strip 14c, the bottom and side attachment strips 34 and 38 can comprise a conventional adhesive such as glue or rubber cement, a mechanical or hook attachment such as a VELCRO™ strip, or any other non-permanent arrangement allowing for adhesion or mechanical positioning of the insert 10c.

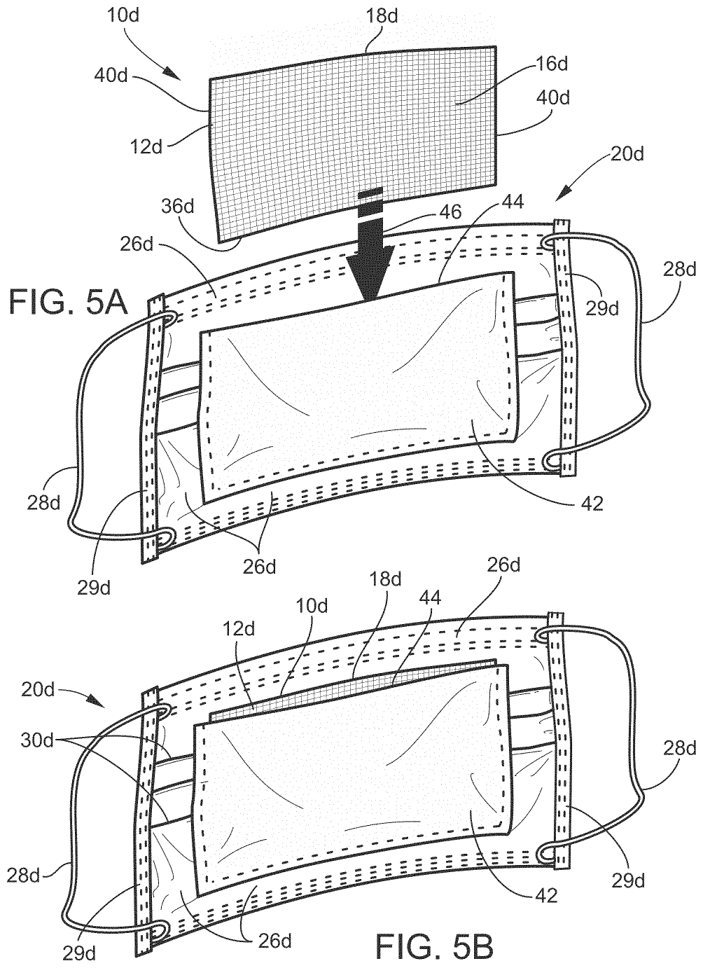

It will be further appreciated that inserts of the invention can also be positioned and attached to masks in other ways within the contemplated scope of the invention. For example, FIGS. 5A and 5B depict a rear view of an insert 10d and face mask 20d of the invention, the face mask 20d having a pocket 42 open at the pocket top 44. The pocket 42 is in most contemplated embodiments constructed of a material that is either identical to the rest of the face mask 20d or, alternatively, of a material that is about at least equally air permeable. The insert 10d is shown prior to attachment to the mask 20d in FIG. 5A as it is moved in a downward direction 46 toward the open pocket top 44.

As is best understood by comparing FIGS. 5A and B, the bottom edge 36d of the insert 10d is then inserted through the open pocket top 44, the pocket 42 itself being sufficiently sized to accommodate both side edges 40d of the insert 10d and to allow encapsulation of most or all of the remaining dimensions of the insert body 12d for attachment and correct positioning and alignment during use. The open pocket top 44 may also include an inside overflap (not shown in FIGS. 5A and B) to fit over the top edge 18d of the insert body 12d for further securement while the insert 10d is attached to the mask 20d. It will be appreciated that such attachment method would be particularly useful for embodiments where the mask 20d is reusable or where the mask material is suited for surface adhesion or attachment.

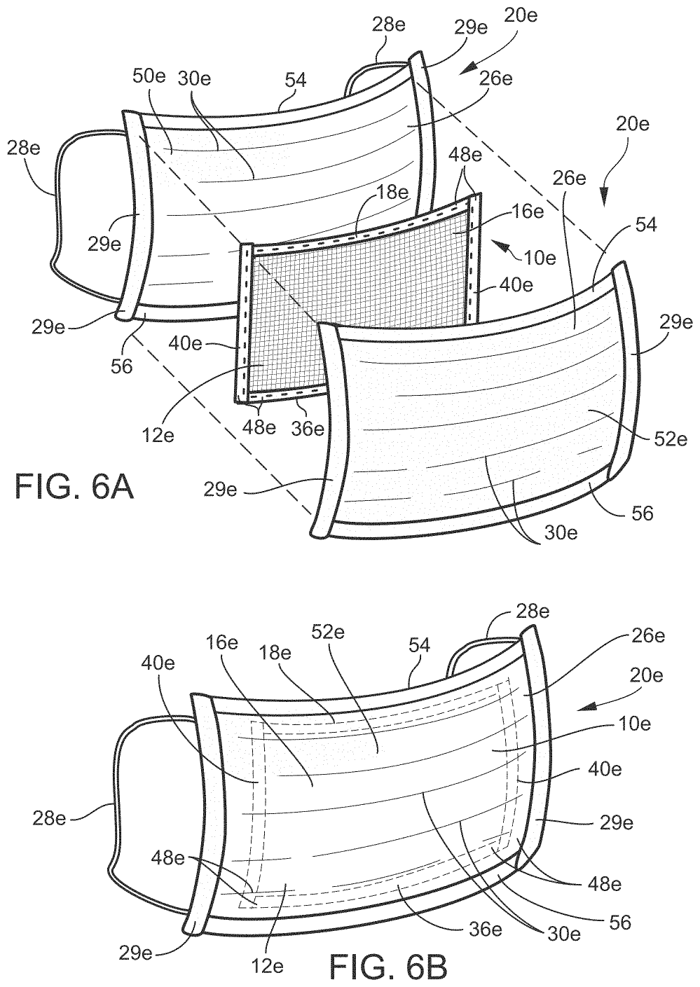

Other mask configurations for positioning an insert by encapsulating the insert within a mask are also within the intended scope of the invention. For example, FIG. 6A depicts an exploded front view of a face mask 20e and insert 10e of the invention wherein the insert includes overfolds 48e along the top edge 18e, bottom edge 36e, and side edges 40e. The mask body 26e is divided into an inside ply 50 and an outside ply 52, each of the plies 50 and 52 having a top fold 54, bottom fold 56, and side folds 29e to form an outer perimeter of each ply 50 and 52. Each of the overfolds 48e of the inside ply 50 allows for attachment to the overfolds 48e of the outside ply 52 using a surface adhesion such as VELCRO™ strips or a removable or a chemical adhesive such as glue or rubber cement to connect the overfolds 48e along their lengths to bind the separate plies 50 and 52 of the mask 20e together, as best understood by comparing the exploded view of FIG. 6A with the assembled view of the mask 20e and insert 10e of FIG. 6B. When the mask 20e and insert 10e are assembled as depicted in FIG. 6B, the inside and outside plies 50 and 52 are sized to allow the top, bottom, and side edges 18e, 36e, and 40e of the insert body 12e to fit inside the attached sidefolds 29e of the mask 20e to allow the insert 10e to be fully encapsulated between the plies 50 and 52 securely positioned within the mask 20e for use.

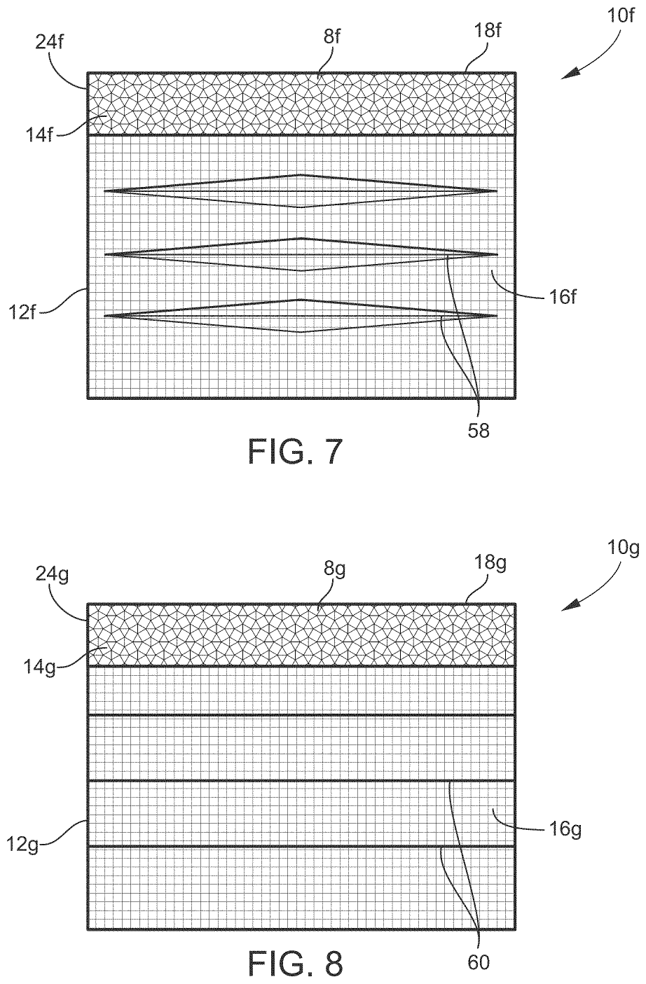

Although the invention has been shown and described as using mask inserts constructed of flat and flexible pieces of copper mesh, it will be appreciated that the flexibility of the cidal metal or cidal metal alloy materials comprising the major structural component of an insert can be stiffened or manipulated by incorporating different folding, pressing, stamping, or bending techniques within the contemplated scope of the invention. For example, FIG. 7 depicts a front view of an insert 10f of the invention having multiple pleats 58 added to the copper mesh of the insert body 12f which extend horizontally and partially along the width of the insert body 12f and filtering portion 16f. In addition to increasing the overall stiffness of the insert 10f, the pleats 58 also provide additional angled surface area to allow increased air interaction with the filtering portion 16f and copper ions in the mesh of the insert body 12f to enhance cidal action and air purification.

Other stiffening insert manipulations are also possible. For example, FIG. 8 depicts a front view of an insert 10g of the invention having multiple folds 60 added to the copper mesh of the insert body 12g. The folds 60 extend horizontally along the full width of the insert body 12g and filtering portion 16g of the insert 10g. Like the pleats 58 in FIG. 7, the folds 60 in FIG. 8 also increase the overall stiffness of the insert 10g. The folds 60 further provide an easily manufactured means for stiffening the insert 10g while providing additional layering of copper mesh for the insert body 12g and filtering portion 16g. Such additional layering allows increased air interaction with the filtering portion 16g and copper ions in the mesh of the insert body 12g to enhance cidal action and air purification.

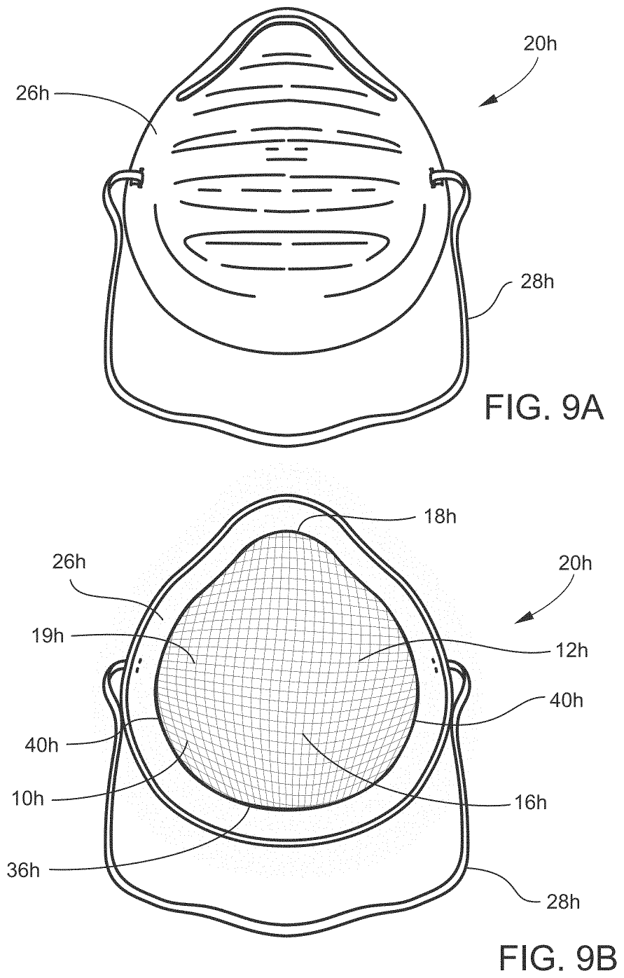

Although the invention has been shown and described for use with flexible flat face masks, it will be appreciated the invention can also be implemented in other styles of masks, including molded and non-flat masks, within the contemplated scope of the invention. For example, FIG. 9A depicts a front view of a conventional, respirator-style of face mask 20h having a single flexible head strap 28h and a pre-molded, non-flat shape to accommodate a wearer's face. FIG. 9B depicts a rear view of the mask 20h of FIG. 9A having an attached, shape-formed insert 10h. The insert 10h includes an insert body 12h and filtering portion 16h constructed of cidal metal or cidal metal alloy that is both the major structural component of the insert 10h and formed, stamped, or molded to fit inside of and match the pre-molded or non-flat shape of the mask 20h. The matched shaping of the insert body 12h along its top edge 18h, bottom edge 36h, and side edges 40h with the pre-molded, non-flat shaping of the mask 20h allows for both proper alignment/positioning and attachment of the insert 10h to the mask 20h.

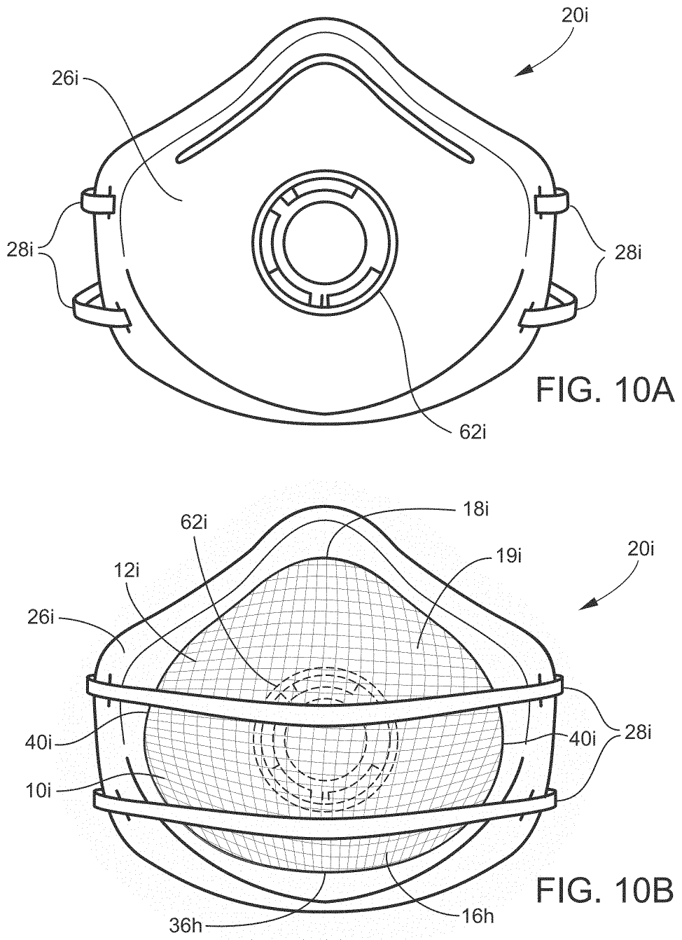

It will be further appreciated the invention can also be implemented in face masks that utilize additional filtration apparatuses. For example, FIG. 10A depicts a filtering facepiece respirator-style of face mask 20i having an additional filtration apparatus 62 in addition to double flexible head straps 28i and a pre-molded, non-flat shape to accommodate a wearer's face. FIG. 10B depicts a rear view of the mask 20i of FIG. 10A having an attached, shape-formed insert 10i. The insert 10i includes an insert body 12i and filtering portion 16i constructed of cidal metal or cidal metal alloy that is both the major structural component of the insert 10i and formed, stamped, or molded to fit inside of and match the pre-molded or non-flat shape of the mask 20i . The matched shaping of the insert body 12i along its top edge 18i, bottom edge 36i, and side edges 40i with the pre-molded, non-flat shaping of the mask 20i allows for both proper alignment/positioning and attachment of the insert 10i to the mask 20i.

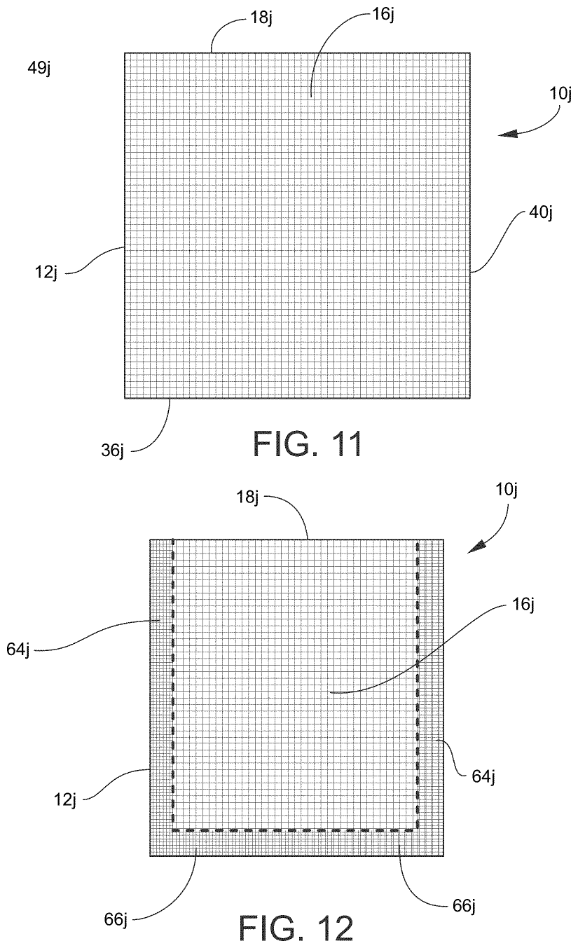

It will also be appreciated that inserts of the invention can be modified and sized to optimize wearer comfort. For example, FIG. 11 depicts an insert 10j of the invention having an insert body 12j and filtering portion 16j comprising a square of cidal metal or cidal metal alloy mesh having a top edge 18j, side edges 40j, and bottom edge 36j. Empirical testing has shown that such a square shape is optimal when precision cut to a 3-inch square for an adult-sized insert and 2.5-inch square for a child-sized insert, as such insert body sizes do not result in heat build-up and allow for suitable modification for further comfort.

For example, as best understood by comparing FIG. 11 with FIG. 12, such modification for an 3-inch square adult-sized insert involves folding the insert body 12j at side edges 40j inwardly to create side folds 64j and further folding the insert body 12j at bottom edge 36j inwardly to create a bottom fold 66j. Inward folding of side edges 40j and bottom edge 36j has been shown to be optimal when the resultant side folds 64j and bottom fold 66j have widths of 0.25 inches, reducing the overall width of the insert body 12j to 2.5 inches and the overall length of the insert body 12j to 2.75 inches. Empirically determined optimal modification for a 2.5-inch square child-sized insert also involves inward folding of side edges 40j and bottom edge 36j where the resultant side folds 64j and bottom fold 66j also have widths of 0.25 inches, reducing the overall width of the insert body 12j to 2.0 inches and the overall length of the insert body 12j to 2.25 inches. The side of the insert body 12j depicted in FIG. 12 now having the side folds 64j and bottom fold 66j thus becomes the back of the insert 10j.

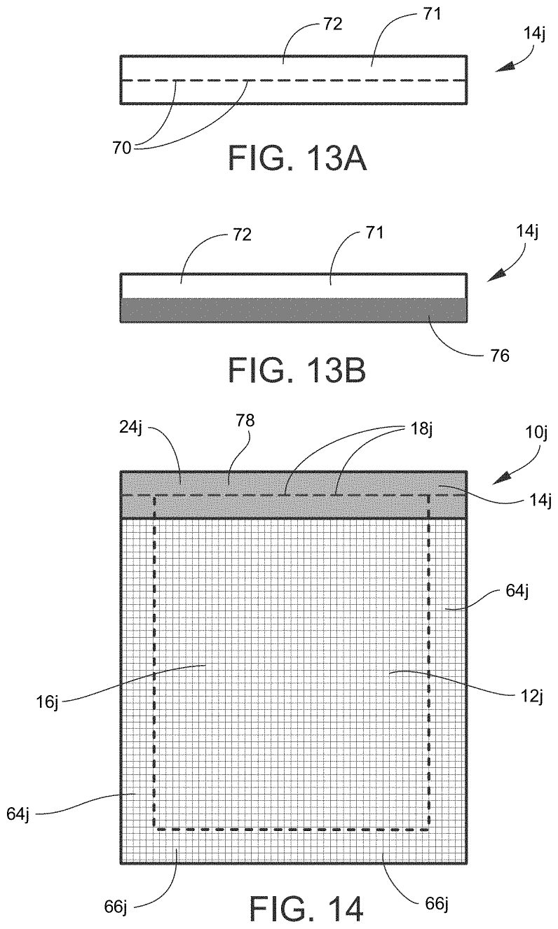

As best understood by comparing FIGS. 12 with FIGS. 13A through 14, the addition of a suitable and optimally comfortable top attachment strip 14j to the insert 10j involves the scoring of the top attachment strip 14j lengthwise along a score line 70 to separate a paper backing on the back surface 71 of the strip 14j into a top paper segment 72 and bottom paper segment 74 as depicted in FIG. 13A. While bisecting the paper backing lengthwise, the scoring does not extend beyond the paper backing into the top attachment strip 14j itself. Now comparing FIGS. 13A and B, this allows for removal of the bottom paper segment 74 while allowing the top paper segment 72 to remain in place, exposing adhesive material only on the bottom half of the top attachment strip 14j as depicted in FIG. 13B.

FIG. 14 depicts a front view of the insert 10j, as modified in FIG. 12. Comparing FIGS. 13A, 13B, and 14, the top attachment strip 14j is flipped over as depicted in FIG.14. This allows the adhesive exposed on the back surface 71 of the strip 14j, after removal of the bottom paper segment 74, to adhere to the front of the insert body 12j near and along the top edge 18j as shown in FIG. 14. The front surface 78 of the top attachment strip 14j is a soft moleskin that allows for optimal wearer comfort. In this position, the top paper segment 72 remains attached to the top attachment strip 14j, just above the top edge 18j of the insert body 12j, to prevent exposure of the adhesive material 76. The top paper segment 72 then remains in place until the wearer removes the top paper segment 72 to allow the insert 10j to be affixed to a mask.

Empirical testing has shown that for an insert 10j of FIGS. 12 and 14 that is adult-sized, the top attachment strip 14j will optimally add 0.25 inches of length to the insert 10j for an overall adult-sized insert length of 3.0 inches. Empirical testing has further shown that for an insert 10j of FIGS. 12 and 14 that is child-sized, the top attachment strip 14j will optimally add 0.25 inches of length to the insert 10j for an overall child-sized insert length of 2.5 inches.

Those skilled in the art will realize that this invention is capable of embodiments different from those shown and described. It will be appreciated that the detail of the structure of the disclosed apparatuses and methodologies can be changed in various ways without departing from the invention itself. Accordingly, the drawings and Detailed Description of the Invention are to be regarded as including such equivalents as do not depart from the spirit and scope of the invention.

1. A removable insert for a face mask, said insert comprising:
an insert body, said insert body being constructed primarily of a material that includes a cidal metal or cidal metal alloy wherein said cidal metal or cidal metal alloy is the major structural component of said insert body;
said insert body being positionable on the face mask wherein said insert body is positioned to cover at least a portion of the wearer's mouth, nose, or mouth and nose when the mask is worn on the wearer's face; and
said insert having a filtering portion, said filtering portion comprising cidal metal mesh or cidal metal alloy mesh for providing cidal action, air purification, and self-disinfection.

2. The insert of claim 1 wherein said filtering portion kills fungi, pathogens, and microorganisms.

3. The insert of claim 1 wherein said filtering portion filters dust and particles.

4. The insert of claim 1 wherein said insert is attached to the mask with an adhesive strip.

5. The insert of claim 1 wherein said insert is attached to the mask with a VELCRO™ strip.

6. The insert of claim 1 further comprising a perimeter barrier around the outer edge of said mask insert body.

7. The insert of claim 1 further comprising pleats on said filtering portion.

8. The insert of claim 1 further comprising folds on said filtering portion.

9. The insert of claim 1 wherein said major structural component of said insert body is flexible mesh.

10. The insert of claim 1 wherein said major structural component is formed into a flat piece of flexible cidal metal or cidal metal alloy mesh.

11. The insert of claim 1 wherein said major structural component is formed into a contoured piece of flexible cidal metal or cidal metal alloy mesh.

12. The insert of claim 1 wherein said filtering portion is removable from the mask.

13. The insert of claim 1 wherein said filtering portion has an average wire diameter and an average width of opening of sufficient size to prevent the penetration of water through said filtering portion.

14. The insert of claim 1 wherein said filtering portion has an average wire diameter and an average width of opening of sufficient size to allow the penetration of disinfecting solution that is isopropyl alcohol solution through said filtering portion.

15. The insert of claim 1 wherein said cidal metal or cidal metal alloy is at least one of copper, silver, gold, bronze, brass, copper alloy, gold alloy, silver alloy, or exotic cidal alloy.

16. The insert of claim 1 wherein said insert body is positionable between two plies of the mask.

17. The insert of claim 1 wherein said insert body is positionable within a pocket of the mask.

18. The insert of claim 1 wherein said average wire diameter of said mesh is about 0.0045 inches.

19. The insert of claim 1 wherein said average wire diameter of said mesh is between about 0.0014 and 0.0045 inches.

20. The insert of claim 1 wherein said average wire diameter of said mesh is less than about 0.0070 inches.

21. The insert of claim 1 wherein said average wire diameter of said mesh is less than about 0.0100 inches.

22. The insert of claim 1 wherein said average width openings of said mesh is between about 0.0070 and 0.00555 inches.

23. The insert of claim 1 wherein said average width openings of said mesh is less than about 0.0100 inches.

24. A removable insert for a face mask, said insert comprising:
an insert body, said insert body being constructed primarily of a material that includes a cidal metal or cidal metal alloy wherein said cidal metal or cidal metal alloy is the major structural component of said insert body;
said insert body being positionable on the face mask wherein said insert body is positioned to cover at least a portion of the wearer's mouth, nose, or mouth and nose when the mask is worn on the wearer's face;
said insert having a filtering portion, said filtering portion comprising cidal metal mesh or cidal metal alloy mesh for providing cidal action, air purification, and self-disinfection;
said filtering portion having an average wire diameter and an average width of opening of sufficient size to prevent, due to water surface tension, the penetration of water through said filtering portion; and
said filtering portion having an average wire diameter and an average width of opening of sufficient size to allow, due to disinfecting solution surface tension that is less than water surface tension, the penetration of disinfecting solution through said filtering portion.

25. The insert of claim 24 wherein said filtering portion kills fungi, pathogens, and microorganisms.

26. The insert of claim 24 wherein said filtering portion filters dust and particles.

27. The insert of claim 24 wherein said insert is attached to the mask with an adhesive strip.

28. The insert of claim 24 wherein said insert is attached to the mask with a VELCRO™ strip.

29. The insert of claim 24 further comprising a perimeter barrier around the outer edge of said insert body.

30. The insert of claim 24 further comprising pleats on said filtering portion.

31. The insert of claim 24 further comprising folds on said filtering portion.

32. The insert of claim 24 wherein said major structural component of said insert body is flexible mesh.

33. The insert of claim 24 wherein said major structural component is formed into a flat piece of flexible cidal metal or cidal metal alloy mesh.

34. The insert of claim 24 wherein said major structural component is formed into a contoured piece of flexible cidal metal or cidal metal alloy mesh.

35. The insert of claim 24 wherein said filtering portion is removable from said mask.

36. The insert of claim 24 wherein said filtering portion has an average wire diameter and an average width of opening of sufficient size to prevent the penetration of water through said filtering portion.

37. The insert of claim 24 wherein said filtering portion has an average wire diameter and an average width of opening of sufficient size to allow the penetration of disinfecting solution that is isopropyl alcohol solution through said filtering portion.

38. The insert of claim 24 wherein said cidal metal or cidal metal alloy is at least one of copper, silver, gold, bronze, brass, copper alloy, gold alloy, silver alloy, or exotic cidal alloy.

39. The insert of claim 24 wherein said insert body is positionable between two plies of the mask.

40. The insert of claim 24 wherein said insert body is positionable within a pocket of the mask.

41. The insert of claim 24 wherein said average wire diameter of said mesh is about 0.0045 inches.

42. The insert of claim 24 wherein said average wire diameter of said mesh is between about 0.0014 and 0.0045 inches.

43. The insert of claim 24 wherein said average wire diameter of said mesh is less than about 0.0070 inches.

44. The insert of claim 24 wherein said average wire diameter of said mesh is less than about 0.0100 inches.

45. The insert of claim 24 wherein said average width openings of said mesh is between about 0.0070 and 0.00555 inches.

46. The insert of claim 24 wherein said average width openings of said mesh is less than about 0.0100 inches.

47. A face mask comprising:
A mask body and a removable insert having an insert body, said insert body being constructed primarily of a material that includes a cidal metal or cidal metal alloy wherein said cidal metal or cidal metal alloy is the major structural component of said insert body;
said insert being positioned to cover at least a portion of the wearer's mouth, nose, or mouth and nose when said mask is worn on the wearer's face; and
said insert having a filtering portion, said filtering portion comprising cidal metal mesh or cidal metal alloy mesh for providing cidal action, air purification, and self-disinfection.

48. The mask of claim 47 wherein said filtering portion of said insert kills fungi, pathogens, and microorganisms.

49. The mask of claim 47 wherein said filtering portion of said insert filters dust and particles.

50. The mask of claim 47 wherein said insert is attached to said mask body with an adhesive strip.

51. The mask of claim 47 wherein said insert is attached to said mask body with a VELCRO™ strip.

52. The mask of claim 47 further comprising a perimeter barrier around the outer edge of said insert body.

53. The mask of claim 47 further comprising pleats on said filtering portion of said insert.

54. The mask of claim 47 further comprising folds on said filtering portion of said insert.

55. The mask of claim 47 wherein said major structural component of said insert body is flexible mesh.

56. The mask of claim 47 wherein said major structural component of said insert body is formed into a flat piece of flexible cidal metal or cidal metal alloy mesh.

57. The mask of claim 47 wherein said major structural component of said insert body is formed into a contoured piece of flexible cidal metal or cidal metal alloy mesh.

58. The mask of claim 47 wherein said filtering portion of said insert body is removable from said mask body.

59. The mask of claim 47 wherein said filtering portion of said insert body has an average wire diameter and an average width of opening of sufficient size to prevent the penetration of water through said filtering portion.

60. The mask of claim 47 wherein said filtering portion of said insert body has an average wire diameter and an average width of opening of sufficient size to allow the penetration of disinfecting solution that is isopropyl alcohol solution through said filtering portion.

61. The mask of claim 47 wherein said cidal metal or cidal metal alloy of said insert body is at least one of copper, silver, gold, bronze, brass, copper alloy, gold alloy, silver alloy, or exotic cidal alloy.

62. The mask of claim 47 wherein said insert body is positioned between two plies of said mask.

63. The mask of claim 47 wherein said insert body is positioned within a pocket of said mask.

64. The mask of claim 47 wherein said average wire diameter of said mesh is about 0.0045 inches.

65. The mask of claim 47 wherein said average wire diameter of said mesh is between about 0.0014 and 0.0045 inches.

66. The mask of claim 47 wherein said average wire diameter of said mesh is less than about 0.0070 inches.

67. The mask of claim 47 wherein said average wire diameter of said mesh is less than about 0.0100 inches.

68. The mask of claim 47 wherein said average width openings of said mesh is between about 0.0070 and 0.00555 inches.

69. The mask of claim 47 wherein said average width openings of said mesh is less than about 0.0100 inches.
